(19) United States
(12) Patent Application Publication
(10) Pub. No.: US 2009/0312438 A1
(43) Pub. Date: Dec. 17, 2009

(54) Compositions and Methods for Attenuating the Formation of A2E in the Retinal Pigment Epithelium

(76) Inventors: Paul S. Bernstein, Salt Lake City, UT (US); Prakash Bhosale, Des Moines, IA (US)

Correspondence Address: Davis, Brown, Koehn, Shors & Roberts, P.C.;The Davis Brown Tower, Suite 1300, 215 10th Street, Des Moines, IA, 50309, US

(21) Appl. No.: 12/483,411

(22) Filed: Jun. 12, 2009

Related U.S. Application Data
(60) Provisional application No. 61/131,885, filed on Jun. 13, 2008.

(51) Int. Cl. A61K 31/047; A61P 27/02; A61P 27/12

(52) U.S. Cl. 514/729

(57) ABSTRACT

A method of decreasing the content of 2-(2,6-dimethyl-8-(2,6,6-trimethyl-1-cyclohexen-1-yl)-1E,3E,5E,7E-octatetraenyl)-1-(2-hydroxyethyl)-4-(4-methyl-6-(2,6,6-trimethyl-1cyclohexen-1-yl)-1E,3E,5E-hexatrienyl-pyridinium (A2E) and its isomers in a subject by administering a therapeutically effective amount of a macular carotenoid formulation.

### CROSS REFERENCE TO RELATED APPLICATIONS

This application claims priority to U.S. Patent Application Ser. No. 61/131,885, filed Jun. 13, 2008, which is incorporated herein in its entirety by this reference.

### BACKGROUND OF THE INVENTION

The present invention relates generally to compositions and methods for attenuating the formation of 2,6-dimethyl-8-(2,6,6-trimethyl-1-cyclohexen-1-yl)-1E,3E,5E,7E-octatetraenyl)-1-1(2-hydroxyethyl)-4-4(4-methyl-6-(2,6,6-trimethyl-1-cyclohexen-1-yl)-1E,3E,5E-hexatrienyl-pyridinium (A2E) in the retinal pigment epithelium and, more specifically to the administration of therapeutically effective amounts of xanthophylls, including but not limited to lutein and zeaxanthin, to reduce levels of A2E in persons at risk for eye disease.

Age-related macular degeneration (AMD) is the major cause of irreversible blindness in developed countries, yet its molecular pathophysiology remains inadequately understood (1). Cellular damage due to high levels of oxidative stress appears to be one of the main pathological explanations for age-related ocular diseases including AMD (2), and cellular accumulation of lipofuscin, a complex mixture of highly fluorescent retinoid and phospholipid metabolites, is considered to be a primary pathogenic biomarker of aging in the retinal pigment epithelium (RPE) (3).

A2E is the major fluorophore identified in the RPE. Chemically, it is a combination of two all-trans-retinal molecules and one ethanolamine molecule (4). Levels of A2E and other lipofuscin components rise with age, with light exposure, and with development of AMD, and early onset macular disorders such as Stargardt and Best diseases are notable for unusually high levels of A2E in humans and in animal models (5-9). Studies have demonstrated that A2E and its cis isomers can act as blue-light-mediated photosensitizers for the generation of reactive oxygen species that could cause damage and cell death in the macula, potentially leading to loss of central vision (10-11).

On the other hand, the dietary xanthophyll carotenoids lutein and zeaxanthin are concentrated at very high levels in the human macula and to a lesser extent in the peripheral retina where they are believed to limit retinal oxidative damage by absorbing incoming blue light and/or by quenching reactive oxygen intermediates (12-13). In vitro studies have suggested that the ocular carotenoids may alleviate A2E-mediated oxidative damage either by direct quenching or by screening phototoxic blue light (14), but in vivo evidence is notably lacking, in part due to the difficulty in obtaining human ocular tissues and the rarity of non-primate small animal models that accumulate significant levels of both A2E and ocular carotenoids. Here we report the relationship of A2E and carotenoids in the macula and peripheral retinal of a large collection of human eyes, and we study the inhibition of A2E formation by dietary carotenoids in the Japanese quail Coturnix japonica, a bird that has substantial ocular levels of both A2E and carotenoids.

### SUMMARY OF THE INVENTION

A2E and its isomer iso-A2E, major fluorophores of lipofuscin in the human retinal pigment epithelium (RPE), are thought to be important mediators of light-induced oxidative damage associated with aging and other ocular disorders. In order to assess the potential protective effect of macular carotenoids against this damage, ocular carotenoids in overlying retinal tissues were measured by HPLC coupled with mass spectral detection (HPLC-MS) and were correlated with the levels of A2E. A statistically significant increase in the total A2E levels in the human RPE/choroid with age (N=66, P,0.0001) was found. Mass spectral peaks at m/z 608 and 624, presumed monofuranoids and monoperoxides of A2E, were detected exclusively in aged donors. In addition, A2E levels in the macular region (2.7±1.1 ng/8-mm punch, n=31) were found to be approximately one third lower than in peripheral retinal regions of same size (9.1±4.6 ng/8-mm punch, n=31). There was a statistically significant inverse correlation between peripheral retina carotenoids and peripheral RPE/choroid A2E and iso-A2E. Prospective carotenoid supplementation studies in Japanese quail (Coturnix japonica) demonstrated nearly complete inhibition of A2E formation. Individuals at risk for visual loss from AMD can reduce the effects by taking a nutritional supplement that contains macular carotenoids.

### BRIEF DESCRIPTION OF THE DRAWINGS

FIGS. 1A-F are HPLC PDA-chromatograms (A and B) and full scan mass spectra (C, D, E, F) of the A2E and iso-A2E peaks from the RPE/choroids of a 14-year-old (A, C, & E) and a 74-year-old (B, D, & F) donor.

FIGS. 2A-C are graphical representations of the age-wise distribution of A2E levels in human whole RPE/choroid (A); 8-mm macular punches (B); and 8-mm peripheral retina punches (C). There was a significant increase observed with age (P<0.001) in all cases.

FIGS. 3A and 3B are graphical representations of the distribution of RPE/choroid A2E levels in relation to overlying retinal carotenoid levels in 8-mm macular punches (A) and 8-mm peripheral retina punches (B). A statistically significant inverse correlation between carotenoids in the peripheral retina and A2E levels in the underlying RPE-choroid (R=−0.36, N=40, P=0.02) however, this inverse relationship was not statistically significant between macular carotenoids and macular A2E (R=−0.28; N=35; P=0.10).

FIG. 4 is a graphical representation of the manipulation of ocular carotenoid and A2E levels in response to dietary supplementation with lutein or zeaxanthin in Japanese quail for 16 weeks (N=4 for all groups). In the upper panel, dark grey bars represent total carotenoids, and light grey bars represent total lutein and zeaxanthin content in the respective groups. Control animals were on a low carotenoid diet for 16 weeks. The A2E levels in the RPE were significantly higher for the control group relative to the other three groups (P<0.001). The A2E levels of other groups were not significantly different from each other.

FIGS. 5A-D are graphical representations of the full scan mass spectra of the A2E extracted from RPE of experimental birds. Week 1 control diet (A); Week 10 control diet (B); Week 12 zeaxanthin-supplemented diet (C) Week 12 lutein-supplemented diet (D).

### DETAILED DESCRIPTION OF PREFERRED EMBODIMENTS

### Definitions

As used herein, the term “eye disease” refers to either an acute or chronic condition, which may in some instances be a result of oxidation processes, including macular degeneration, age-related macular degeneration; Stargardt disease, Best diseases, retinitis pigmentosa, and cataracts.

“Visual performance” refers to the visual performance of a subject. Visual performance includes visual acuity, low contract acuity, mesopic acuity, letter contract sensitivity, grating contrast sensitivity, disability glare, intraocular stray light, and visual fields. An improvement in visual performance includes an improvement in an aspect of vision, such as an improvement in visual acuity, low contract acuity, mesopic acuity, letter contract sensitivity, grating contrast sensitivity, or visual fields or a reduction in disability glare or intraocular stray light.

“Macular carotenoid formulation” refers to a composition that includes carotenoids found in the macula of the eye, principally lutein and zeaxanthin.

As used herein, the term “therapeutically effective amount” refers to the amount/dose of a compound or pharmaceutical composition that is sufficient to produce an effective response (i.e., a biological or medical response of a tissue, system, animal or human sought by a researcher, veterinarian, medical doctor or other clinician) upon administration to a subject. The “therapeutically effective amount” will vary depending on inter alia the disease and its severity, and the age, weight, physical condition and responsiveness of the subject to be treated.

As used herein, the terms “treated” and “treating” refers to preventing or delaying the appearance of clinical symptoms of a disease or condition in a subject that may be afflicted with or predisposed to the disease or condition, but does not yet experience or display clinical or subclinical symptoms of the disease or condition. “Treating” also refers to inhibiting the disease or condition, i.e., arresting or reducing its development or at least one clinical or subclinical symptom thereof. “Treating” further refers to relieving the disease or condition, i.e., causing regression of the disease or condition or at least one of its clinical or subclinical symptoms. The benefit to a subject to be treated is either statistically significant or at least perceptible to the subject and/or the physician.

### Formulations

The invention provides the use as above wherein the composition is for administration as a unit dose. In another embodiment of the invention, the unit dose contains the active ingredient(s) in an amount from about 10 μg/kg to 10 mg/kg body weight, in another embodiment from about 25 μg/day/kg to 1.0 mg/day/kg, in yet another embodiment from about 0.1 mg/day/kg to 1.0 mg/day/kg body weight. In another embodiment, the unit dose is containing the active ingredient in an amount from 0.1 mg/day/kg to 1.0 mg/day/kg body weight.

According to the invention, the compounds mentioned above may be administered in any suitable way e.g. orally or parenterally, and it may be presented in any suitable form for such administration, e.g. in the form of tablets, capsules, powders, syrups or solutions or dispersions for injection. In another embodiment, and in accordance with the purpose of the present invention, the compound of the invention is administered in the form of a solid pharmaceutical entity, suitably as a tablet or a capsule or in the form of a suspension, solution or dispersion for injection. The compound of the invention is most conveniently administered orally in unit dosage forms such as tablets or capsules, containing the active ingredient in an amount from about 10 μg/kg to 10 mg/kg body weight, for example 25 μg/day/kg to 1.0 mg/day/kg.

Compounds of the present invention may be administered as an oral dose form, such as a solid oral dose form, typically tablets or capsules, or as a liquid oral dose form, or may be administered in an immediate release dosage form or a controlled or sustained release dosage form. The compounds may be conveniently administered orally in unit dosage forms, such as tablets or capsules, containing the active ingredient in an amount from about 0.1 to about 150 mg/day, from about 0.2 to about 100 mg/day, from about 0.5 to about 50 mg/day, from about 0.1 to about 50 mg/day, from about 1 to about 15 mg/day, or from about 2 to about 5 mg/day. Typically, the pharmaceutical composition comprises from about 0.5 mg to about 20 mg, such as about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg or about 20 mg of one or more of the compounds.

In one embodiment, the compound(s) of the present invention are administered once daily (for example, in the morning or afternoon) using doses of about 2.5 mg to about 20 mg. In another embodiment, the compound(s) are administered in a more prolonged and continuous release, e.g., administration 2-3 times daily with low doses or a modified release formulation prepared using conventional methods known in the art, such that about 5 to about 50 mg administered to the subject per 24 hour period.

According to the present invention, the compound(s) of the present invention or a pharmaceutically acceptable salt thereof may be administered in any suitable way, e.g., orally or parenterally, and it may be presented in any suitable form for such administration, e.g., in the form of tablets, capsules, powders, syrups or solutions or dispersions for injection, or as an inhalant. In another embodiment, and in accordance with the purpose of the present invention, the compound(s) of the present invention are administered in the form of a solid pharmaceutical entity, suitably as a tablet or a capsule or in the form of a suspension, solution or dispersion for injection. Additionally, the compound(s) of the present invention may be administered with a pharmaceutically acceptable carrier, such as an adjuvant and/or diluent.

Methods for the preparation of solid or liquid pharmaceutical preparations are well known in the art. See, e.g., Remington: The Science and Practice of Pharmacy, 21st ed., Lippincott Williams & Wilkins (2005). Tablets may thus be prepared by mixing the active ingredients with an ordinary carrier, such as an adjuvant and/or diluent, and subsequently compressing the mixture in a tabletting machine. Non-limiting examples of adjuvants and/or diluents include: corn starch, lactose, talcum, magnesium stearate, gelatine, lactose, gums, and the like. Any other adjuvant or additive such as colorings, aroma, and preservatives may also be used provided that they are compatible with the active ingredients. The pharmaceutical compositions of the invention thus typically comprise an effective amount of the compound(s) of the present invention and a pharmaceutically acceptable carrier.

The compounds may be administered systemically in a form selected from the group consisting of: an aerosol suspension of respirable particles; a liquid or liquid suspension for administration as nose drops or nasal spray; a nebulized liquid for administration to oral or nasopharyngeal airways; an oral form; an injectable form; a suppository form; and a transdermal patch or a transdermal pad.

One such means involve an aerosol mixture of respirable particles comprised of the active compounds, which the subject inhales. The therapeutic compound is absorbed into the bloodstream via the lungs in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Ever since its identification as a major fluorophore of ocular lipofuscin, A2E has been considered an important mediator of pathalogical processes involved in aging and retinal degenerations (3-6). Clinically, autofluorescent compounds in the human retinal pigment epithelium increase with age when measured with autofluorescence imaging (17-18), and early onset macular dystrophies such as Stargardt and Best diseases likewise exhibit high levels of lipofuscin deposition (7, 9). In dry age-related macular degeneration, intense autofluorescence is often seen at the border of geographic atrophy in regions in which further expansion of atrophy is likely to occur (19). Increased levels of A2E have been confirmed biochemically in autopsy eyes of elderly individuals and in donor eyes from individuals with macular dystrophies (4-9). Animal models of dominant and recessive Stargardt disease have been developed that accumulate A2E such as the Abca4 knockout mouse (heterozygous and homozygous) as well as the Elov14 transgenic mouse (7-8). In vitro studies indicate that A2E may exert its toxic effects on the RPE through blue-light-mediated free radical generation or by induction of lysosomal dysfunction through detergent-like and pH altering effects (10-11, 20-21).

There is general consensus that limiting A2E formation is a worthwhile target for pharmacological interventions against AMD and related diseases, and a number of groups in academia and industry have dedicated themselves to this task. A2E formation has been linked to intense light exposure which causes high degrees of throughput of retinoids in the visual cycle, facilitating formation of elevated amounts of various Schiff base adducts of all-trans-retinal with phosphatidylethanolamine, the precursors of A2E (22-23). Although severe light restriction inhibits A2E formation in animal models (22), this approach is probably not practical in humans. Therefore, inhibitors of the visual cycle have been a primary focus. These include 13-cis-retinoic acid, an FDA approved acne medicine that inhibits dark adaptation through alcohol dehydrogenase and/or isomerase inhibition (24), fenretinide, a retinoid analogue that induces a moderate systematic deficiency of vitamin A (25), and RPE-65 antagonists targeted to inhibit a key step of the vertebrate mechanism which isomerizes all-trans-retinoids to 11-cis-retinoids (26). All of these agents will cause some degree of night blindness which may be uncomfortable for the patients, and retinoid-based compounds when used chronically may cause significant systemic side effects and teratogenicity. Non-retinoid RPE-65 antagonists appear to be well tolerated in animal models, but there is little, if any, human experience with these compounds.

The macular carotenoids lutein and zeaxanthin have also been considered as possible antagonists against the formation and the toxic effects of A2E (14). Multiple epidemiological studies have demonstrated an inverse correlation between high dietary intakes, blood levels, and macular levels of these xanthophylls and risk of AMD (27-30), and the AREDS 2 study is currently evaluating their efficacy against AMD in a large, randomized, placebo-controlled, prospective manner. The macular carotenoids efficiently absorb blue light, the region of the visible spectrum that is most likely to produce free radicals from A2E, and in vitro they can inhibit photo-oxidation of A2E and its precursors (14).

### Materials and Methods

### Chemicals

Organic solvents were HPLC grade from Fisher Scientific (Hampton, N.H.). Standards of A2E and iso-A2E were prepared and column purified in the laboratory of Dr. Heidi R. Volmer-Snarr, Department of Chemistry and Biochemistry, Brigham Young University, Provo, Utah. They were dissolved in methanol (MeOH) at a concentration of 1 μg/ml, stored at −70° C., and brought to room temperature before use. The concentration of the stock solution of A2E was confirmed spectroscopically [E (IM) at 439 nm=36900] using a published extinction coefficient (4). Similar standard stock solutions of lutein (Kemin Health, Des Moines, Iowa) and zeaxanthin (DSM, Kaiseraugst, Switzerland) were prepared, and concentrations were confirmed using published extinction coefficients (33).

### Tissue Procurement and Processing

Human donor eyes were obtained from the Utah Lions Eye Bank within 24 hours after death after corneas had been harvested for transplantation. None of the donors had a known history of eye disease. Tissue procurement and distribution complied with the tenets of the Declaration of Helsinki. The time between donor death and enucleation was not more than four hours. Dissections were carried out 6-24 hours after donor death in a dim light environment. These data exclude older outliers of age 48 or greater who had been consuming high-dose lutein supplements regularly prior to death (34). All eyecups were visually inspected with a handheld magnifier to exclude the presence of obvious ocular pathology such as intermediate or large drusen, hemorrhages, or scars. After carefully removing adherent vitreous, macular and mid-peripheral retinal tissues were excised with an 8-mm circular trephine. The underlying RPE/choroid layer was then carefully excised using the same trephine. Diverse non-human mammalian eyes were obtained from neighboring laboratories or local slaughterhouses. Since non-primate animal eyes do not have a macula, the entire retina and RPE/choroid were isolated and processed for small eyes, and the 8-mm trephine was used in a random region for large eyes. We weights were recorded for all collected tissues after blotting excess moisture. Total protein levels and protein separation patterns determined by Bradford assay and by one-dimensional SDS gel electrophoresis, respectively, were similar in both macular and retinal RPE/choroid punches from humans (˜250μ/8-mm of tissue).

Extraction of A2E from RPE/Choroid

A2E and its isomers were extracted and isolated from RPE/choroid using a previously described method (8). RPE samples were homogenized in 1:1 CHCl₃/MeOH (2 ml) and 0.01 M phosphate-buffered saline (PBS) (1 ml). The homogenizer was washed with 1:1 CHCl₃/MeOH (2 ml), 0.01 M PBS (1 ml), and then CHCl₃ (2 ml) and CH₂Cl₂ (2 ml) were added to remove any remaining material. All solutions were combined, and the organic layer was extracted from the aqueous layer. The combined organic extracts were evaporated to dryness under vacuum at room temperature. The residue was dissolved in MeOH for HPLC. The vials were centrifuged at approximately 2000 g to remove the minor amounts of insoluble solid particles prior to analysis.

Extraction of Carotenoids from Retina

Tissues were homogenized and extracted three times with tetrahydrofuran containing 0.1% butylated hydroxytoluene by sonication at 5° C. to 10° C. for 30 minutes each time. The combined organic extracts were evaporated to dryness under vacuum at room temperature. The dried residue was redissolved in one ml of HPLC mobile phase and centrifuged at approximately 2000 g for 10 minutes to remove the minor amounts of insoluble solid particles prior to analysis. The majority of carotenoids in bird retinas are esterified (32), so after the initial extraction, the dried carotenoid residue was redissolved in hexane and subjected to saponification in 1.8% (w/v) methanolic potassium hydroxide (KOH) for two hours at room temperature. After saponification, the samples were washed with water until the samples achieved neutral pH. The vials were centrifuged at approximately 2000 g to remove the minor amounts of insoluble solid particles. The solution was evaporated to dryness on a rotary evaporator under reduced pressure at room temperature and reconstituted in the appropriate HPLC solvents.

### HPLC Conditions

HPLC analysis was performed on a Thermo Separations (San Jose, Calif.) HPLC system with binary gradient pumps, a refrigerated autosampler, a UV6000 photodiode-array detector (PDA), and an MSQ single quadrupole mass spectrometer. Peak identities were confirmed by PDA and mass spectra and by co-elution with authentic standards as necessary. Calibration was by external standardization curves with authentic standards. We do not routinely use internal standards because they may interfere with low-level analytes in small biological samples (35). Typical reproducibility with external standardization in our laboratory is +5%.

A2E HPLC analysis: The dried A2E samples were redissolved in 100 μl of MeOH. A gradient of 84-100% acetonitrile (A) with 0.05% trifluoroacetic acid in H₂O (B) over 35 minutes was used to separate A2E at a flow rate of 1.0 ml min⁻¹ on a reverse-phase C18 column (4.6×250 mm, Phenomenex, Atlanta, Ga.). The column was maintained at room temperature, and the HPLC PDA detector was operated at 440 nm.

Carotenoid HPLC analysis: The dried extracts were re-dissolved in 100 μl of HPLC mobile phase [hexane:dichloromethane:methanol: N,N-di-isopropylethylamine (80:19.2:0.7:0.1 v v⁻¹)]. HPLC separation was carried out at a flow rate of 1.0 ml min⁻¹ on a cyano column (Microsorb 25 cm length×4.6 mm id, particle size 5 μm, Rainin Instrument Co., Woburn, Mass.). The column was maintained at room temperature, and the HPLC PDA detector was operated at 450 nm.

### Mass Spectometry (MS) Equipment and Conditions

MS analysis was performed using a Thermo Separations (San Jose, Calif.) MSQ single quadrupole mass spectrometer, equipped with an electron spray ionization (ESI) source and an atmospheric pressure chemical ionization (APCI) source. A2E and carotenoids were ionized in positive ion ESI and APCI modes, respectively. To avoid overloading of eluted solvent molecules in the mass spectrometer and to optimize ionization conditions, 50% of the eluate was directed to waste with the help of a diverter valve after the PDA detector. The delay time from PDA to MS was 0.13 minutes. The protonated precursor molecular ions were initially acquired in full-scan mode from 300-1000 Da with 0.2 step size, revealing the molecular masses of the components. Selected ion monitoring (SIM) was performed using dwell time of 200 ms for each channel. In SIM mode, the m/z channels 592±3, 608±1.5, 624±1.5, and 640±1.5 were used for A2E and its oxidative products. Typical detection conditions for A2E were: RF lens bias voltage 0.1V, cone voltage 80V and heater temperature 550° C. The ion source and tuning lens parameters were optimized automatically by infusing A2E samples via the injector. For carotenoids, the m/z channels 551±0.7 and 569±0.7 were used for lutein, and 569±0.8 for zeaxanthin. Typical detection conditions were: corona discharge current 5 μA, cone voltage 80V, and probe temperature 500° C.

### Xanthophyll Supplementation Studies in Japanese Quail

Adult Japanese quail (Coturnix japonica) were procured from B&D farms (Harrah, Okla.), and all experiments were reviewed and approved by the University of Utah's Institutional Animal Care and Use Committee. The birds were kept under ambient room light on a 12-hour light-dark cycle, and they were divided into three groups of four birds: lutein supplemented, zeaxanthin supplemented, and unsupplemented. All birds were fed daily with a standard turkey diet (˜20 g/day) (Purina Mills, St. Louis, Mo.) that was low in carotenoids (1.10 ng/g). The supplemented birds were gavaged daily with 0.5 ml of a microbial extract rich in lutein or zeaxanthin for 16 weeks (0.2 mg of carotenoid per bird per day). Lutein was prepared from the freshwater alga Chlorella protothecoides CS 41 (Microalgae Supply Service, CSIRO, Hobart Australia) under conditions described earlier (36). Zeaxanthin was obtained from the non-pathogenic bacteria Flavobacterium multivorum ATCC 55238 grown on liquid growth medium as detailed earlier (37). After the experiments, the birds were sacrificed, and the A2E and carotenoid content of the RPE/Choroid and retina, respectively, were measured as described above.

### Statistics

Reported values are mean±standard deviation (SD). Statistical analysis was done using Microcal Origin version 6.0 (Northampton, Mass.). In most cases, a two-population (independent) t-test was performed with significance level set at 0.05.

### Results

### A2E and Carotenoid Levels in Human RPE/Choroid and Retina

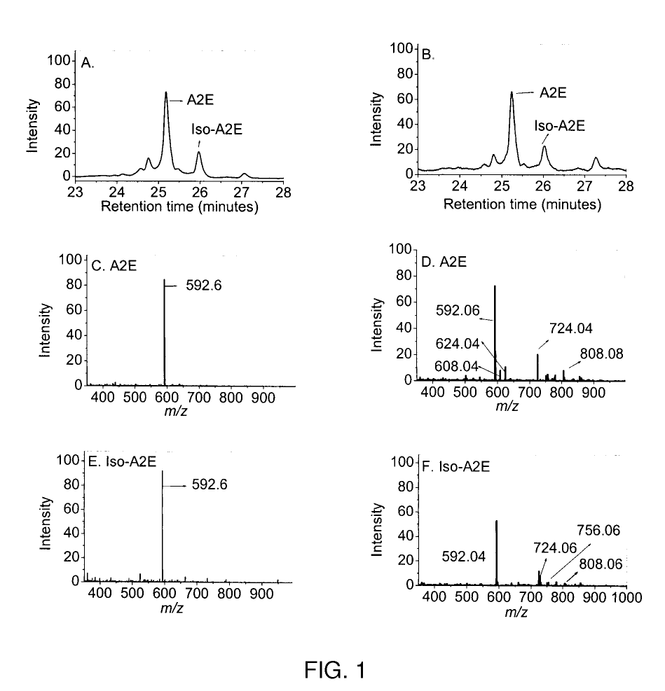

We used photodiode array (PDA) and mass-spectral (MS) detection for convenient in-line HPLC quantitation and identification of A2E and its isomers from human RPE/choroid. Younger human eyes (<50 years old; N=22) always had a single major molecular ion peak for A2E and iso-A2E at m/z 592.6, while eyes older than 50 (N=31) were more likely to exhibit detectable higher molecular weight ions of A2E, two of which are consistent with previously reported oxidation products at m/z 608 and 624 (N=24) (15-16), as well as unidentified higher m/z ions between 724 and 808 (FIG. 1). Single ion monitoring (SIM) of these ions allowed the quantification these peaks relative to the A2E peak at m/z 592±3, 608±1.5, and 624±1.5. The ratio of the m/z 608 and 624 peaks present in donors above 50 years relative to unoxidized A2E was 0.027±0.008 (N=24), while these oxidized peaks were absent in the donors below age 50.

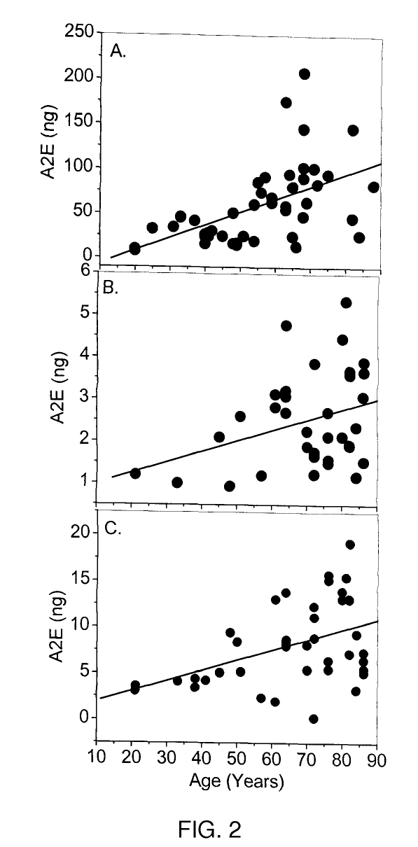

Initial experiments with whole RFP/choroid from human eyes between ages 20 to 88 revealed a linear increase of A2E with age (N=66, P<0.0001) (FIG. sA). This same significant increase with age was present in both the macula and the peripheral retina, although A2E levels were typically 4-fold higher in the periphery (FIGS. 2B and 2C).

We speculated that lower levels of macular A2E might be related to higher levels of carotenoids in the overlying retina. Table 1 shows that this was indeed the case with 10-fold higher levels of total carotenoids in the macula versus the periphery and greater than 3-fold higher levels of A2E in the periphery versus the macula in young and old eyes.

TABLE 1
A2E levels in the RPE/Cchoroid and carotenoids in
the overlying retina in 8-mm macular and peripheral
punches from human eyes (mean ± SD)*.

| | Donors <50 | Donors >50 | |
|---|---|---|---|
| | years old | years old | Statistical |
| | (N = 22) | (N = 31) | significance |
| Age (Years) | 37 ± 10 | 74 ± 9 | P < 0.0001 |
| A2E in macular (ng) | 1.3 ± 0.5 | 2.7 ± 1.1 | P < 0.001 |
| A2E in periphery (ng) | 5.1 ± 2.6 | 9.1 ± 4.6 | P < 0.0001 |
| Iso-A2E in macula (ng) | 0.1 ± 0.1 | 0.8 ± 0.4 | P < 0.01 |
| Iso-A2E in periphery (ng) | 0.5 ± 0.2 | 1.4 ± 0.9 | P < 0.0001 |
| Total carotenoids in | 43.6 ± 14.6 | 38 ± 15.1 | P < 0.001 |
| macula (ng) | | | |
| Total carotenoids in | 2.6 ± 2.2 | 2.8 ± 2.8 | Not |
| periphery (ng) | | | significant |

*These data exclude older outliers of age 48 or greater who had been consuming high-dose lutein supplements regularly prior to death (34).

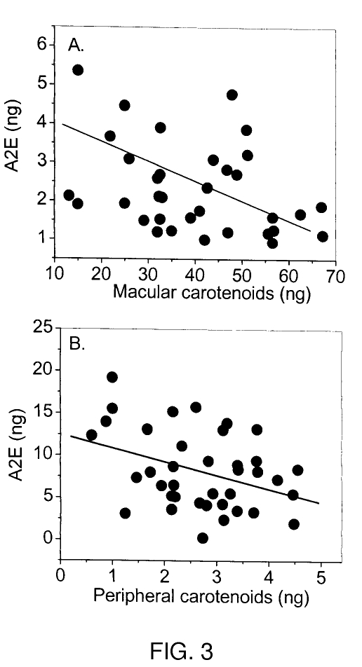

Retinal carotenoid levels were inversely correlated with A2E levels in the underlying RPE/choroid in both the macula (R=−0.28; N=35; P=0.10) and the peripheral retina (R=−0.36; N=40, P=0.20), but only the peripheral retina reached statistical significance (FIG. 3).

### A2E and Carotenoid Levels in Animal RPE/Choroid and Retina

The human eye is not easily amenable to experimental manipulation of carotenoid and A2E levels due to the slow changes of macular carotenoid levels in response to dietary manipulations and the invasive nature of the A2E analytical measurements, so we surveyed the eyes of a variety of higher vertebrates and compared ocular carotenoid and A2E levels on a wet weight basis (Table 2).

TABLE 2
A2E and carotenoid levels in the RPE/choroid and overlying
retina in a variety of higher vertebrates.

| | | | | | Total retinol |
|---|---|---|---|---|---|
| | | | N | RPE/choroid | carotenoid |
| Animal | Age | | (eyes) | A2E content | content |
| Young human | | | | | |
| a. macula (8-mm punch) | 23 ± 5 | years | 22 | 26 ± 12 | 611.1 ± 19.6 |
| b. periphery (8-mm punch) | | | | 102 ± 16 | 91.1 ± 15 |
| Older human | | | | | |
| a. macula (8-mm punch) | 60 ± 6 | years | 31 | 53.4 ± 13 | 716.6 ± 12.9 |
| b. periphery (8-mm punch) | | | | 182 ± 23 | 118.5 ± 42 |
| Young monkey | | | | | |
| Macaca fascicularis | | | | | |
| a. macula (8-mm punch) | 2 ± 2 | years | 8 | 73 ± 5 | 401.1 ± 15.1 |
| b. periphery (8-mm punch) | | | | 53 ± 85 | 9.5 ± 3.2 |
| Older monkey | | | | | |
| Macaca fascicularis | | | | | |
| a. macula (8-mm punch) | 9 | years | 4 | 86 ± 15 | 323.1 ± 33.1 |
| b. periphery (8-mm punch) | | | | 93 ± 85 | 3.5 ± 5.2 |
| Marmoset | 2 ± 1 | years | 8 | 187.5 ± 1.2 | 211.1 ± 24.2 |
| Callithrix jacchus | | | | | |
| Cow | Adult | | 2 | 16 ± 2.8 | 30.3 ± 12.1 |
| Pig | Adult | | 2 | 23 ± 3.2 | 2.3 ± 2.5 |
| Rat * | 3 | months | 12 | 0.029 ± 0.007 | 0.002 ± 0.004 |
| Mouse * | 3 ± 3 | months | 16 | 0.034 ± 0.002 | 0.006 ± 0.0019 |
| Young Japanese quail | | | | | |
| a. female | 8 ± 2 | weeks | 8 | 19.3 ± 3.2 | 43.2 ± 10 |
| b. male | | | 8 | 24.2 ± 7.2 | 66.2 ± 12 |
| Older Japanese quail | | | | | |
| a. female | 26 ± 4 | weeks | 8 | 838 ± 4.6 | 32.4 ± 11.0 |
| b. male | | | 8 | 42 ± 2.3 | 33.7 ± 12.0 |

* For rats and mice, pooled samples of n = 6 and n = 8 were used for extraction and detection of both carotenoids and A2E.

As expected, monkey eyes most closely resembled human eyes with respect to A2E and carotenoid content, but these laboratory animals are expensive to procure and difficult to manage and handle. Young rat and mouse eyes contain barely detectable levels of carotenoids, and there is no published literature demonstrating that high-dose carotenoid supplementation can alter these very low levels. Moreover, their A2E levels relative to tissue wet weight are orders of magnitude lower than the other animals listed. Generally, rodents have high levels of A2E only when they are appropriately genetically modified, as in the Abca4 knockout and Elov14 transgenic mice (7-8). Cows and, to a lesser extent, pigs have reasonable levels of A2E and carotenoids suitable for dietary manipulation, but these animals are quite large. The female Japanese quail possesses the best combination of small size and reasonable levels of ocular A2E and carotenoids, so they were selected for further study.

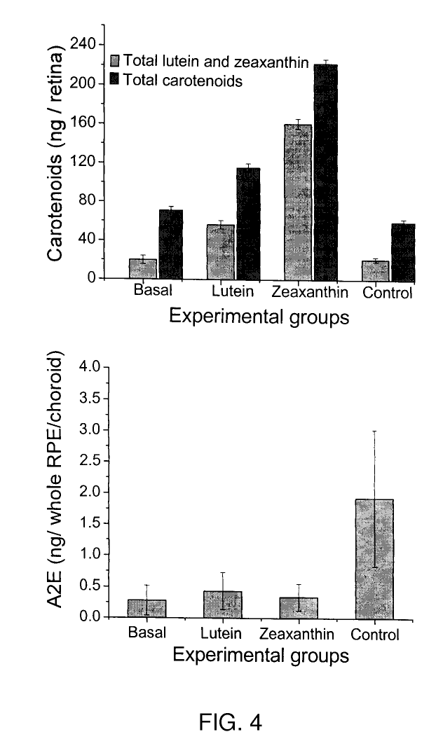

Young adult female Japanese quail were supplemented with (3R, 3′R, 6′R)-lutein or (3R, 3′R)-zeaxanthin for a 16-week period and compared to unsupplemented quail. As shown in FIG. 4 and Table 3, total retinal carotenoid levels rose 1.6-fold in the lutein-supplemented group and 3.1-fold in the zeaxanthin supplemented group relative to baseline, while control diet birds decreased 16%. Total lutein and zeaxanthin levels increased 2.8-fold in the lutein supplemented group and 8.1-fold in the zeaxanthin supplemented groups relative to baseline, while control diet birds increased 6.5%. All of these increases in supplemented birds were statistically significant (P<0.05). Supplementation of lutein and zeaxanthin also led to significant increases in the levels of lutein, zeaxanthin and their carotenoid metabolites in the RPE in comparison to basal and control groups (P<0.05) (Table 3).

TABLE 3
Carotenoid content in the RPE/choroid and retina of Japanese quail at the
beginning and end of the supplementation experiments.

| | Carotenoids | | | | | | | |
|---|---|---|---|---|---|---|---|---|
| | in | | | | Carotenoids | | | |
| | RPE/choroid | | | | in retina | | | |
| | at baseline | Carotenoids in | | | At baseline | Carotenoids in | | |
| | (ng/tissue ± | RPE/choroid at 16 weeks | | | (ng/tissue ± | retina at 16 weeks | | |
| | SD; N = 4) | (ng/tissue ± SD; N = 2) | | | SD; N = 4) | (ng/tissue ± SD; N = 4) | | |
| | Basal | Control | Lutein | Zeaxanthin | Basal | Control | Lutein | Zeaxanthin |
| Adonirubin | 0.23 ± 0.42 | 0.3 ± 0.51 | 0.32 ± 0.21 | 0.61 ± 0.07 | 3.4 ± 3.1 | 1.9 ± 1.5 | 2.4 ± 2.1 | 6.4 ± 3.9 |
| 3′-Oxolutein | 0.31 ± 0.09 | — | 0.15 ± 0.12 | 1.6 ± 1.0 | 3.8 ± 3.2 | 3.1 ± 1.1 | 3.8 ± 3.1 | 5.9 ± 2.3 |
| Lutein | 2.8 ± 1.0 | 0.25 ± 0.1 | 1.2 ± 0.3 | 1.5 ± 0.31 | 5.1 ± 3.2 | 3.4 ± 3.2 | 32.2 ± 6.3 | 3.9 ± 2.6 |
| Astaxanthin | 0.2 ± 0.5 | 5.1 ± 0.6 | 5.2 ± 0.9 | 1.6 ± 15 | 18.2 ± 1.5 | 10.6 ± 2.3 | 31.2 ± 4.5 | 16.2 ± 3.5 |
| Zeaxanthin | — | 1.72 ± 0.3 | 3.1 ± 0.4 | 3.8 ± 0.9 | 14.8 ± 5.1 | 17.8 ± 1.1 | 24.4 ± 2.1 | 156.8 ± 1.1 |
| B-apo-2′- | — | — | — | — | 6.5 ± 3.5 | 9.6 ± 4.5 | 6.7 ± 4.5 | 8.7 ± 6.5 |
| carotenol | | | | | | | | |
| Unidentified | — | — | — | — | 2.9 ± 2.2 | 4.2 ± 2.4 | 3.9 ± 2.4 | — |
| ε,ε,-Carotene | | — | — | — | 11.5 ± 4.3 | 3.5 ± 6.3 | 14.5 ± 6.5 | 12.5 ± 6.3 |
| Galloxanthin | 0.2 ± 5.1 | — | 2.8 ± 2.9 | 1.2 ± 1.2 | 9.2 ± 4.1 | 9.2 ± 3.2 | 03.2 ± 6.5 | 25.2 ± 3.1 |
| Total | 3.5 ± 3.1 | 7.4 ± 0.35 | 12.8 ± 0.2 | 10.3 ± 0.8 | 75.4 ± 3.6 | 63.3 ± 3.2 | 122.3 ± 4.3 | 235.6 ± 4.6 |
| carotenoids | | | | | | | | |

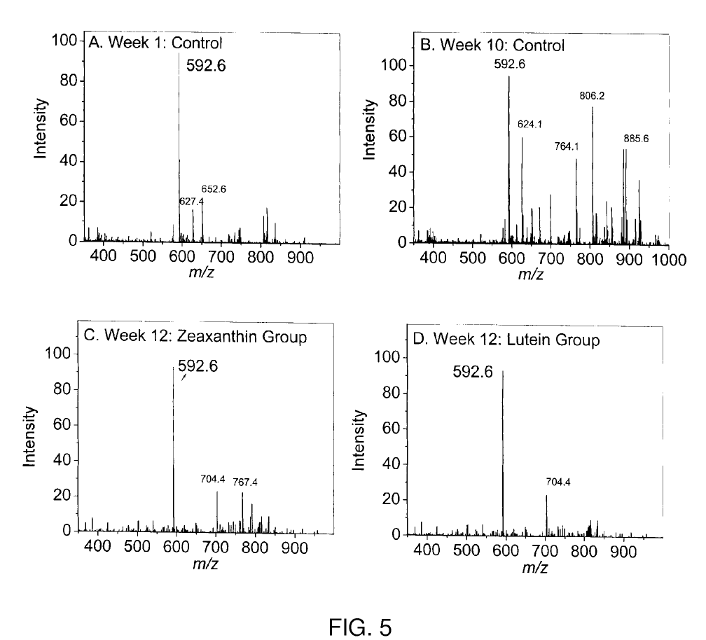

A2E levels rose more than six-fold relative to basal levels (P<0.001) in the unsupplemented control group in a 16-week period, while the lutein and zeaxanthin supplemented birds registered barely any rise from baseline levels (P=0.1). Mass spectra of A2E from control group had higher levels of oxidation products in comparison to mass spectra from the lutein and zeaxanthin supplemented groups (FIG. 5).

### Discussion

In this study, we first examined the relationship between retinal carotenoids and A2E in human donor eyes. We confirmed that A2E rises with age in both the macula and the peripheral retina, and that it becomes more oxidized with age. Interestingly, macular A2E levels in these 8-mm punches were about three-fold lower in the macular relative to the periphery despite its focused light exposure and high metabolic activity. In both the macula and the periphery, there was an inverse correlation between A2E levels and the total carotenoids, although statistical significance was reached only in the periphery. Taken in sum, these human cadaver eye data are consistent with the hypothesis that retinal carotenoids inhibit formation and oxidation of A2E in the underlying RPE, but proof of this hypothesis requires either prospective clinical studies or appropriate animal experiments. Human studies would be challenging because noninvasive quantitative A2E measurements are difficult to execute with adequate reliability, and it is not possible to measure peripheral carotenoids in living humans. Therefore, we turned to animal models for further study.

Rodents have been used most frequently to study inhibitors of A2E formation, but their low endogenous ocular levels of A2E and carotenoids and their inability to incorporate carotenoids into the retina in response to supplementation make further study impossible. We surveyed a diverse array of other potential study animals and determined that the Japanese quail was the best small nonprimate animal model to study further since it had baseline levels of carotenoids and A2E comparable to a human on a wet weight basis, and it is amendable to dietary manipulation of ocular carotenoid levels (31-32). Our experiments demonstrated a profound inhibition of A2E formation in birds supplemented with high dose lutein or zeaxanthin relative to birds fed a control diet low in carotenoids. Since the majority of carotenoids in the quail retina are sequestered as fatty acid esters in oil droplets, it is more likely that inhibition of A2E formation by ingested carotenoids is mediated by a light filtering effect rather than a direct antioxidant mechanism because although there was a significant increase in RPE carotenoids in response to supplementation, levels still remained about ten times lower than the overlying retina.

Our findings provide evidence for a new mechanism for the potential protective effect of lutein and zeaxanthin in degenerative eye disorders—inhibition of A2E formation and oxidation. As noninvasive quantitation of lipofuscin improves, this hypothesis can be directly tested in living humans. Lutein and zeaxanthin are particularly attractive agents for clinical use as A2E formation inhibitors because, unlike the other inhibitors currently under investigation, they are already extensively used in humans as dietary supplements, and no toxicity has ever been reported. Moreover, our investigations point out the value of non-rodent small animal models for testing A2E formation antagonists. At 150-200 grams, an adult Japanese quail is the size of a rat, is easy to care for, and forms several-fold increased levels of A2E over a several month period when fed a control diet low in carotenoids. Thus, rapid screening of novel pharmacological inhibitors of A2E formation should be quite feasible.

### REFERENCES

The following references are incorporated herein in their entirety by this reference:
1. Rattner A, Nathans J (2006) Macular degeneration: recent advances and therapeutic opportunities. Nat Rev Neurosci 7:860-872.
2. Beatty S, Koh H, Phil M, Henson D, Boulton M (2000) The role of oxidative stress in the pathogenesis of age-related macular degeneration. Surv Opthalmol 45:1.15-134.
3. Dorey C K, Wu G, Ebenstein D, Garsd A, Weiter J J (1989) Cell loss in the aging retina. Relationship to lipofuscin accumulation and macular degeneration. Invest Opthalmol Vis Sci 30:1691-4 699.
4. Parish C A, Hashimoto M, Nakanishi K, Dillon J, Sparrow J R (1998) Isolation and one-step preparation of A2E and iso-A2E, fluorophores from human retinal pigment epithelium. Proc Natl Acad Sci USA 95:14609-14613.
5. Lamb L E, Simon J D (2004) A2E: a component of ocular lipofuscin. Photochemistry and Photobiology 79:127-136.
6. Sparrow J R, Boulton M. (2005) RPE lipofliscin and its role in retinal pathobiology. Exp Eye Res 80:595-606.
7. Mata N L, Weng J, Travis G H (2000) Biosynthesis of a major lipofuscin fluorophore in mice and humans with ABCR-mediated retinal and macular degeneration. Proc Natl Acad Sci USA 97:7 154-7 159.
8. Karan G, Lillo C, Yang Z, Cameron D J, Locke K G, Zhao Y, Thirumalaichary S, Li C, Birch D G, Voilmer-Snan H R, Williams D S, Zhang K (2005) Lipofuscin accumulation, abnormal electrophysiology, and photoreceptor degeneration in mutant ELOVL4 transgenic mice: a model for macular degeneration. Proc Natl Acad Sci USA 102:4164-4 169.
9. Bakall B, Radu R A, Stanton J B, Burke J M, McKay B S, Wadelius C, Mullins R F, Stone E M, Travis G H, Marmorstein A D (2007) Enhanced accumulation of A2E in individuals homozygous or heterozygous for mutations in BEST1 (VMD2). Exp Eye Res 85:34-43.
10. Rozanowska M, Wessels J, Boulton M, Burke J M, Rodgers M A, Truscott T G, Sarna T (1998) Blue light-induced singlet oxygen generation by retinal lipofuscin in non-polar media. Free Radic Biol Med 24:1107-1112.
11. Sparrow J R, Zhou J, Ben-Shabat 5, Volimer H, Itagaki Y, Nakanishi K (2002)
Involvement of oxidative mechanisms in blue-light-induced damage to A2E-laden RPE. Invest Opthalmol Vis Sci. 43:1222-1227.
12. Moeller S M, Jacques P F, Blumberg J B (2000) The potential role of dietary xanthophylls in cataract and age-related macular degeneration. J Am Coil Nut 19:522 S-527S.
13. Krinsky N I, Landrum J T, Bone R A (2003) Biologic mechanisms of the protective role of lutein and zeaxanthin in the eye. Annu Rev Nutr 23:171-201
14. Kim S R, Nakanishi K, Itagaki Y, Sparrow J R (2006) Photooxidation of A2-PE, a photoreceptor outer segment fluorophore, and protection by lutein and zeaxanthin. Exp Eye Res 82:828-839.
15. Jang Y P, Matsuda H, Itagaki Y, Nakanishi K, Sparrow J R (2005) Characterization of peroxy-A2E and furan-A2E photooxidation products and detection in human and mouse retinal pigment epithelial cell lipofuscin. J Biol Chem 280:39732-39739.
16. Radu R A, Mata N L, Bagla A, Travis G H (2004) Light exposure stimulates formation of A2E oxiranes in a mouse model of Stargardt's macular degeneration. Proc Natl Acad Sci USA 101:5928-5933.
17. Delori F C (2004) Autofluorescence method to measure macular pigment optical densities fluorometry and autofluorescence imaging. Arch Biochem Biophys 430:156-162.
18. Sharifzadeh M, Bernstein P S, Gellermann W (2006) Nonmydriatic fluorescence-based quantitative imaging of human macular pigment distributions J Opt Soc Am A 10:2373-23 87.
19. Holz F G, Bindewald-Wittich A, Fleckenstein M, Dreyhaupt J, Scholl H P, Schmitz-Valckenberg S; FAM-Study Group (2007) Progression of geographic atrophy and impact of fundus autofluorescence patterns in age-related macular degeneration. Am J Opthalmol 143: 463-472.
20. Sparrow J R, Parish C A, Hashimoto M, Nakanishi K (1999) A2E, a lipofuscin fluorophore, in human retinal pigmented epithelial cells in culture. Invest Opthalmol Vis Sci 40:2988-2995.
21. De S, Sakmar T P (2002) Interaction of A2E with model membranes. Implications to the pathogenesis of age-related macular degeneration. J Gen Physiol 120:147-157,
22. Mata N L, Tzekov R T, Liu X, Weng J, Birch D G, Travis G H (2001) Delayed dark-adaptation and lipofuscin accumulation in abcr^{+/-} mice: implications for involvement of ABCR in age-related macular degeneration. Invest Opthalmol Vis Sci. 42:1685-1590.
23. Kim S R, Fishkin N, Kong J, Nakanishi K, Allikmets R, Sparrow J R (2004) Rpe65 Leu45OMet variant is associated with reduced levels of the retinal pigment epithelium lipoftiscin fluorophores A2E and iso-A2E. Proc Natl Acad Sci USA 101: 11668-11672.
24. Radu R A, Mata N L, Nusinowitz 5, Liu X, Sieving P A, Travis Gil (2003) Treatment with isotretinoin inhibits lipofuscin accumulation in a mouse model of recessive Stargardt's macular degeneration. Proc Natl Acad Sci USA 100:4742-4747.
25. Radu R A, Han Y, Bui T V, Nusinowitz 5, Bok D, Lichter J, Widder K, Travis Gil, Mata N L (2005) Reductions in serum vitamin A arrest accumulation of toxic retinal fluorophores: a potential therapy for treatment of lipofuscin-based retinal diseases. Invest Opthalmol Vis Sci 46:4393-4401.
26. Maiti P, Kong J, Kim S R, Sparrow J R, Allikmets R, Rando R R (2006) Small molecule RPE65 antagonists limit the visual cycle and prevent lipoftiscin formation. Biochemistry 45:852-860.
27. Eye Disease Case-Control Study Group (1993) Antioxidant status and neovascular age-related macular degeneration. Arch Opthalmol 111: 104-109.
28. Seddon J M, A. U., Sperduto R D, Hiller R, Blair N, Burton T C, Farber M D, Gragoudas E S, Hailer J, Miller D T et a!.; Eye Disease Case-Control Study Group (1994) Dietary carotenoids, vitamins A, C, and E, and advanced age-related macular degeneration. JAMA 272:1413-1420.
29. SanGiovanni J P, Chew E Y, Clemons T E, Ferris F L 3rd, Gensler G, Lindblad A S, Milton R C, Seddon J M, Sperduto R D, Age-Related Eye Disease Study Research Group (2007) The relationship of dietary carotenoid and vitamin A, E, and C intake with age-related macular degeneration in a case-control study: AREDS Report No. 22. Arch Opthalmol 125:1225-1232.
30. Bernstein P S, Zhao D Y, Wintch S W, Ermakov I V, McClane R W, Gellermann W (2002) Resonance Raman measurement of macular carotenoids in normal subjects and in age-related macular degeneration patients. Opthalmology 2002 109:1780-1787.
31. Toyoda Y, Thomson L R, Langner A, Craft N E, Garnett K M, Nichols C R, Cheng K M, Dorey C K (2002) Effect of dietary zeaxanthin on tissue distribution of zeaxanthin and lutein in quail. Invest Opthalmol Vis Sci 43:1210-1221.
32. Bhosale P, Serban B, Zhao D Y, Bernstein P S (2007) Identification and metabolic transformations of carotenoids in ocular tissues of the Japanese quail Coturnix japonica. Biochemistry 46:9050-9057.
33. Britton, G. (1995) UV/Visible Spectroscopy, in: Carotenoids, eds Britton G., Liaaen-Jenson S, Pfander H. (Birkhaeuser, Basel, Switzerland) vol IB, pp 13-62.
34. Bhosale P, Zhao D Y, Bernstein P S (2007) HPLC measurement of ocular carotenoid levels in human donor eyes in the lutein supplementation era. Invest Opthalmol Vis Sci 48:543-549.
35. Bhosale P, Zhao D Y, Serban B, Bernstein P S. (2007) Identification of 3-methoxyzeaxanthin as a novel age-related carotenoid metabolite in the human macula. Invest Opthalmol Vis Sci 48:1435-1440.
36. Bhosale P, Serban B, Bernstein P S (2006) Production of deuterated lutein by Chiorella protothecoides and its detection by mass spectrometric methods. Biotechnol Lett 17:1371-1375.
37. Bhosale P, Teredesai P V, Lihong J, Ermakov I V, Gellermann W, Bernstein P S (2005) Production of deuterated zeaxanthin by Flavobacterium multivorum and its detection by resonance Raman and mass spectrometric methods. Biotechnol Lett 21:1719-1723.

We claim:

1. A method of decreasing the content of 2-(2,6-dimethyl-8-(2,6,6-trimethyl-1-cyclohexen-1-yl)-1E,3E,5E,7E-octatetraenyl)-1-(2-hydroxyethyl)-4-(4-methyl-6-(2,6,6-trimethyl-1cyclohexen-1-yl)-1E,3E,5E-hexatrienyl-pyridinium (A2E) and its isomers in a subject in need thereof, comprising administering a therapeutically effective amount of a macular carotenoid formulation, thereby decreasing the content of A2E.

2. The method of claim 1, wherein the carotenoid is lutein.

3. The method of claim 1, wherein the carotenoid is zeaxanthin.

4. The method of claim 1, wherein the subject is a mammal.

5. The method of claim 1, wherein the subject is a human.

6. The method of claim 1, wherein the composition is given orally.

7. The method of claim 1, wherein the A2E is decreased in the retinal pigment epithelium/choroid.

8. The method of claim 1, wherein the A2E is decreased in the macula.

9. The method of claim 1, wherein the A2E is decreased in the retina.

10. The method of claim 1, wherein the A2E is decreased in the lens.

11. The method of claim 1, wherein lutein is the major carotenoid.

12. The method of claim 1, wherein zeaxanthin is the major carotenoid.

13. A method of decreasing oxidation eye tissue in a subject in need thereof, comprising administering a therapeutically effective amount macular carotenoid formulation, thereby decreasing oxidation in the eye tissue.

14. The method of claim 13, wherein the eye tissue is the macula.

15. The method of claim 13, wherein the subject is at risk for or is being treated for age-related macular degeneration.

16. The method of claim 13, wherein the subject is at risk for or is being treated for Stargardt disease.

17. The method of claim 13, wherein the subject is at risk for or is being treated for retinitis pigmentosa.

18. The method of claim 13, wherein the subject is at risk for or is being treated for cataract development.

19. A method of improving visual performance in a subject in need thereof, comprising administering a therapeutically effective amount macular carotenoid formulation, thereby improving visual performance in the subject.
